(19) United States
(12) Patent Application Publication
(10) Pub. No.: US 2013/0116533 A1
(43) Pub. Date: May 9, 2013

(54) LONG-TERM CUTANEOUS CARDIAC MONITORING

(71) Applicant: BIOTRONIK SE & Co. KG, Berlin (DE)

(72) Inventors: Jie Lian, Beaverton, OR (US); Dirk Muessig, West Linn, OR (US); Volker Lang, Berlin (DE)

(73) Assignee: BIOTRONIK SE & Co. KG, Berlin (DE)

(21) Appl. No.: 13/659,177

(22) Filed: Oct. 24, 2012

Related U.S. Application Data
(60) Provisional application No. 61/555,512, filed on Nov. 4, 2011.

(51) Int. Cl. A61B 5/0408; A61B 5/0452; A61B 5/0432; A61B 5/04; A61B 5/00

(52) U.S. Cl. CPC A61B 5/04085; A61B 5/04087; A61B 5/04012; A61B 5/0006; A61B 5/6832; A61B 5/0432; A61B 5/04525
USPC 600/391; 600/393

(57) ABSTRACT

A system for long-term non-invasive heart monitoring includes (1) a disposable unit that has built-in electrodes, a built-in wire antenna, a watertight chamber that can be opened and closed, and an adhesive surface for cutaneous mounting; (2) an electronic controller that can acquire, process and store physiological signals, and can be fitted into the disposable unit to establish electrical contact with the built-in electrodes; and (3) a portable communication unit that can wirelessly communicate bi-directionally with the electronic controller, and further communicate bi-directionally with a remote service center.

### CROSS-REFERENCE TO RELATED APPLICATIONS

This application claims priority under 35 USC §119(e) to U.S. Provisional Patent Application 61/555,512 filed Nov. 4, 2011, the entirety of which is incorporated by reference herein.

### FIELD OF THE INVENTION

The invention relates to systems suitable for long-term non-invasive heart monitoring.

### BACKGROUND OF THE INVENTION

Conventionally, Holter monitors have been used for non-invasive monitoring of heart rhythm, but the monitoring time is usually limited to 24 to 48 hours, or at most a week or so.

Event recorders and external loop recorders have also been used for ambulatory cardiac monitoring, usually lasting for a week to a month or so. These devices can be programmed to record short episodes of electrocardiograms (ECGs) that are manually triggered by the patient upon experiencing symptoms, or automatically record a limited number of arrhythmia episodes detected by the device.

A long-term continuous ECG recorder (Zio™ patch) has also been developed by iRhythm. This disposable device can be worn by the patient for up to two weeks, and can continuously record an ECG. After finishing the recording, the device is mailed to the service center which downloads the recorded ECG and performs offline ECG analysis.

Holter monitors, wearable event recorders, and external loop recorders are not suitable for long-term monitoring (maximum 1 mo). The electrodes usually require daily change and the wires are cumbersome. Although the Zio™ patch is waterproof and can be worn comfortably by a patient for up to one or two weeks, it is also not designed for long-term monitoring (e.g. >6 mo).

US 2011/0125040 A1 discloses a wireless ECG monitoring system that includes a disposable patch having electrodes and an adhesive surface for attachment to the chest of the patient, an ECG monitor that can be hooked up with the disposable patch, and a cell phone handset that can bi-directionally communicate with the ECG monitor. The system is designed to continuously monitor ECG for at least 24 hours, and repeat ECG monitoring after recharging the battery and using new disposable patches. The system has two major limitations. First, the need to periodically recharge the ECG monitor makes it unsuitable for continuous cardiac monitoring. When the ECG monitor is removed from the patch and recharged (for example when the patient is sleeping), no cardiac monitoring can be performed. Second, when switching from one disposable patch to another, there is no quantitative guidance for the patient to properly place the patch over the chest surface. The patient has to rely on a printed guide to find the location and adjust the orientation of the patch, but there is no feedback to the patient whether the placement of the patch is appropriate. As a result, after switching the disposable patches, a recorded ECG may have low quality and be unusable, or the newly acquired ECG may not be comparable to previous recordings.

The implantable loop recorder, which is used for the diagnosis of unexplained syncope (fainting), and for the detection of atrial fibrillation, is suitable for long-term monitoring (usually >1 year). This is the only device currently available for long-term cardiac monitoring, but it requires an invasive operation to implant the device under the skin.

### SUMMARY OF THE INVENTION

The invention, which is defined by the claims set forth at the end of this document, seeks to provide improved systems for long-term non-invasive monitoring of cardiac functions. An exemplary version of the invention achieves this goal with:
(1) a disposable unit that has at least two built-in electrodes; a built-in wire antenna; a watertight chamber that can be opened and closed; and an adhesive surface;
(2) an electronic controller that can acquire, process and store physiological signals; can be fitted into the disposable unit; and can establish electrical contact with the built-in electrodes and the built-in wire antenna; and
(3) a portable communication unit that can wirelessly communicate bi-directionally with the electronic controller, and further communicate bi-directionally with a remote service center. The electronic controller can also communicate bi-directionally with another programming device.

The aforementioned goal is also achieved by a system for long-term cutaneous cardiac monitoring which includes a disposable unit configured to be adhered to a patient's skin, and an electronic controller detachably connected to the disposable unit. The disposable unit includes:
(1) a substantially flat and flexible skin contacting portions that has at least two pick-up electrode poles arranged to electrically contact a patient's skin at two or more spaced-apart locations,
(2) a wire antenna; and
(3) a watertight chamber attached to or integrated within the flat and flexible skin contacting portion, and being configured to accommodate the electronic controller in a watertight sealed manner, and being able to open and close so as to allow removal and reinsertion of the electronic controller.

On the inside of the chamber, electrical terminals are provided that are electrically connected to the pick-up electrode poles, and that are arranged to be operatively connected to respective ports of the electronic controller. Likewise, a conducting antenna port is provided inside the chamber. The conducting antenna port is electrically connected to the wire antenna and arranged to be operatively connected to a respective antenna port of the electronic controller.

The electronic controller includes a battery with a capacity sufficient to allow the electronic controller to operate for at least one year. The electronic controller has contact ports arranged to make contact to the terminals within the chamber and to thus establish electrical contact with the pick-up electrode poles and the wire antenna, respectively.

The electronic controller is configured to acquire, process, and store the physiological signals to be picked up via the electrode poles and to provide quantitative feedback regarding the signal quality of a signal acquired via the pick-up electrode poles.

The proposed system thus has 3 parts: (1) a disposable unit that has at least two built-in electrodes, a built-in wire antenna, a waterproof chamber that can be opened and closed, and an adhesive surface; (2) an electronic controller that has a battery life of at least 1 year, and which can acquire, process and store the physiological signals, and can be fitted into the disposable unit and establish the electrical contact with the built-in electrodes and the built-in wire antenna; and (3) a portable communication unit that can wirelessly communicate bi-directionally with the electronic controller and further communicate bi-directionally with a remote service center. The electronic controller can also communicate bi-directionally with another programming device. Two of these parts, namely the disposable unit and the electronic controller, are physically connected to each other when in use and form a body-worn monitoring device.

A unique feature of this system is that the disposable unit, the electronic controller and the portable communication unit are physically decoupled but functionally coupled. Long-term non-invasive cardiac monitoring is achieved by connecting these components electrically (between the disposable unit and the electronic controller) and wirelessly (between the electronic controller and the portable communication unit).

The disposable unit can be comfortably worn by the patient for (preferably) 1-2 weeks, though shorter or longer periods are possible. The patient can fit the electronic controller into the disposable unit to complete the circuit for ECG monitoring. The electronic controller also connects to the wire antenna in the disposable patch for wireless communication with the portable communication unit.

The patient can remove the electronic controller from one disposable unit and place it in another disposable unit to continue cardiac monitoring. Upon switching to a new disposable unit, the patient can use the portable communication unit to interrogate the electronic controller to check the quality of the acquired ECG signal. Quantitative feedback on the ECG signal quality, such as the morphological similarity between an acquired ECG waveform and pre-stored ECG templates, as well as the signal-to-noise ratio, can be provided to the patient to guide the proper placement of the disposable unit on the body surface.

The ECG and other diagnostic data stored in the electronic controller can be transmitted to the external portable communication unit periodically, and/or upon the occurrence of one or more triggering events, and the external portable communication unit can in turn relay the information to a remote service center. After data transmission, the electronic controller preferably clears the data memory and continues heart monitoring. The battery of the electronic controller supports the normal operation of the electronic controller for at least 12 months. Therefore, by changing the disposable unit every 1-2 weeks (for example), the system can continuously monitor the cardiac rhythm for at least 12 months.

The electronic controller continuously monitors the skin contact of the electrodes in the disposable unit and communicates the related information to the external portable communication unit, which provides instantaneous feedback for the patient. The electronic controller can alert the patient to reposition or replace the disposable unit upon receiving a warning signal from the electronic controller indicating abnormal skin-electrode contact, or based on a pre-configured schedule.

In an exemplary version of the invention, the electronic controller is further configured to provide quantitative feedback regarding the signal quality of a signal acquired via the pick-up electrode poles. Further, the electronic controller can be configured to maintain at least one representative ECG beat template. In particular, the electronic controller is further configured to construct the representative ECG beat template by averaging multiple normal beats of ECG signals acquired via the pick-up electrode poles, and being aligned with predefined fiducial points. Even more particularly, the electronic controller is configured to start constructing the representative ECG beat template immediately upon affixing the disposable unit on a patient's skin, and to continuously update the representative ECG beat template based on acquired ECG signals.

The disposable unit preferably has three built-in electrode poles. Preferably, these electrode poles are spaced apart from each other by an inter-electrode distance of at least 3 cm.

The electronic controller preferably includes electric components that are hermetically sealed inside a case of the electronic controller. These components preferably include an electronic circuitry including a microprocessor, a memory, and an electronic interface circuitry preferably including a feedthrough circuitry for noise reduction, a high voltage protection circuitry, a switch network circuitry for sensing channel selection, and front-end analog filters, and an ECG sensing unit connected to the electronic interface circuitry and the microcontroller, with the ECG sensing unit including amplifiers, analog-to-digital converters, digital filters, etc.

The electronic controller preferably includes an impedance measuring unit that connects to the conducting ports for connecting the terminals, with the impedance measuring unit being configured to measure an impedance signal between the electrode poles. Thus, the electronic controller can both pick up ECG signals and measure inter-electrode impedance.

It is preferred that the portable communication unit is configured to interrogate the electronic controller to check the morphology and quality of an acquired ECG signal. In particular, it is preferred that the system is configured to wirelessly transmit a latest representative ECG beat template from the electronic controller to the portable communication unit and then compare a newly acquired representative ECG beat template with one or more old representative ECG beat templates that are pre-stored in the portable communication unit.

Preferably the portable communication unit and/or the electronic controller is configured to rate a newly acquired representative ECG beat template as being acceptable only when its morphology is sufficiently similar to at least one of the old representative ECG beat templates stored in the portable communication unit. Alternatively or additionally, the portable communication unit and/or the electronic controller can be configured to rate a newly acquired representative ECG beat template as being acceptable only when it has sufficiently high signal quality, in particular, if the signal-to-noise ratio of an QRS complex in the ECG beat template is greater than a predefined threshold. Alternatively or additionally, the portable communication unit and/or the electronic controller can be configured to rate a newly acquired representative ECG beat template as being acceptable only when its morphology is sufficiently similar to at least one of the old representative ECG beat templates and it has sufficiently high signal quality as measured by the signal to noise ratio.

The portable communication unit and/or the electronic controller is preferably configured to generate a signal indicating a necessity to reposition the disposable unit if the newly acquired representative ECG beat template is rated as being unacceptable. Thus, the system can assist a patient in positioning the disposable unit in case it has to be replaced. Furthermore, consistency is maintained between ECG signals being picked up prior to replacement with ECG signals being picked up after replacement.

### BRIEF DESCRIPTION OF THE DRAWINGS

The foregoing and other features and advantages of the invention will be more apparent from the following detailed description in conjunction with the following drawings, wherein:

FIG. 1A provides a simplified bottom view, and FIG. 1B provides a simplified side view, of a disposable unit 100 and an electronic controller 200 of an exemplary version of the invention;

FIG. 2A illustrates a cover 160 over the top of the disposable unit 100, and FIG. 2B illustrates a cover 160 over the bottom of the disposable unit 100;

FIG. 3A illustrates a bottom view of the disposable unit 100 with its chamber 150 housing the electronic controller 200, and FIG. 3B presents a side view;

FIG. 4 shows a bottom view of a multi-electrode configuration of the disposable unit 100, shown with its corresponding electronic controller detached and spaced from the disposable unit 100;

FIG. 5 schematically illustrates the preferred operation of the long-term cardiac monitoring system;

FIG. 6 schematically illustrates the storage of multiple (preferably at least 3) old representative ECG beat templates in the portable communication unit 600;

FIG. 7 shows a block diagram of an electronic controller 200 connecting with a disposable unit 100 for cutaneous ECG monitoring, and its interfaces with an external programming device 800 and a portable communication unit 600 (which further communicates with a remote service center 700);

FIG. 8 shows the external portable communication unit 600 in conjunction with a button 610 which can be pressed by the patient to trigger an event recording by the electronic controller 200

### DETAILED DESCRIPTION OF PREFERRED VERSIONS OF THE INVENTION

The following description is of preferred versions of the invention, and the invention is not limited to these preferred versions. The scope of the invention should be determined with reference to the claims set forth at the end of this document.

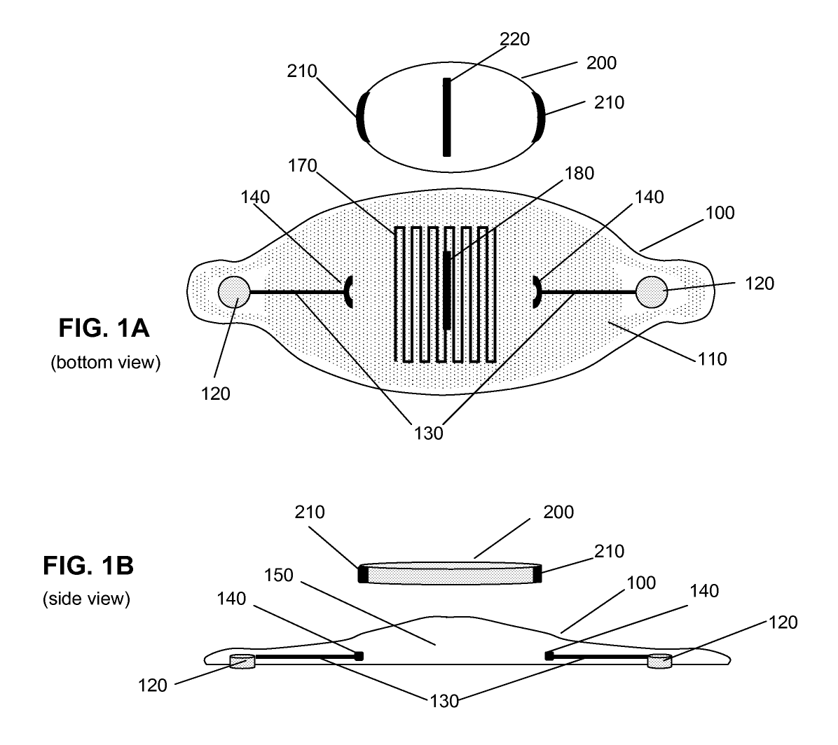

FIG. 1A shows a bottom view, and FIG. 1B shows a side view, of an exemplary disposable unit 100 and electronic controller 200. To achieve long-term non-invasive cardiac monitoring, a lightweight, waterproof and disposable skin-adhesive unit 100 is provided. Because the unit is disposable, it can be easily replaced with another disposable unit after its usage for a few days. The bottom of the disposable unit 100 has an adhesive surface 110 that can be affixed to the patient's skin for at least 7 days. One exemplary type of such adhesive material is the pressure-sensitive adhesive which forms a bond when pressure is applied to stick the adhesive to the adherent (e.g., the patient's skin).

The disposable unit 100 has at least two built-in electrodes 120. Preferably, the inter-electrode distance is greater than 3 cm. The electrodes 120 are embedded inside the disposable unit 100 but protrude from its bottom surface so that they can establish contact with the patient's skin. The electrodes 120 are connected to the respective electrical conducting terminals 140 via the conducting wires 130. Both conducting wires 130 and the electrical conducting terminals 140 are enclosed inside the disposable unit 100 which has a watertight housing that is made of a flexible biocompatible polymer.

Preferably, the disposable unit 100 also has a built-in wire antenna 170. Because of the relatively large surface area of the disposable unit, a long wire antenna 170 (e.g. >6 cm in length) can be embedded, for example by arranging the wire antenna 170 in a serpentine/zigzag pattern or a spiral/multiple loop pattern. The wire antenna 170 also has a conducting port 180. Both the wire antenna 170 and its conducting port 180 are enclosed inside the disposable unit 100.

The electronic controller 200 includes electronic circuitry that is hermetically sealed inside a case which is preferably made from a biocompatible material. At least two electrical conducting ports 210, which are isolated from each other, are embedded on the case of the electronic controller 200. In addition, a separate conducting antenna interface 220 is also embedded on the case of the electronic controller 200. In normal working condition as described hereinafter, the electronic controller 200 preferably has a battery life of at least 1 year without the need of recharging.

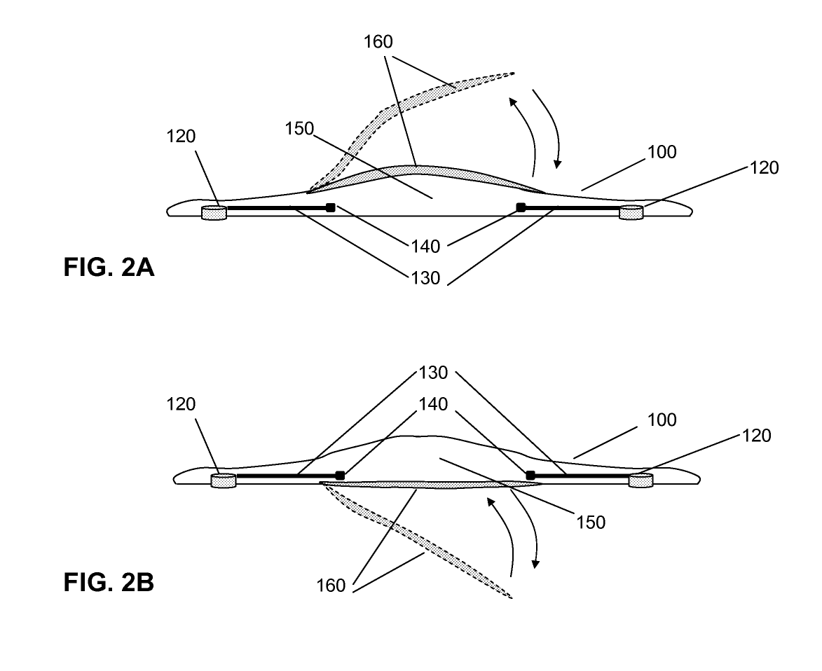

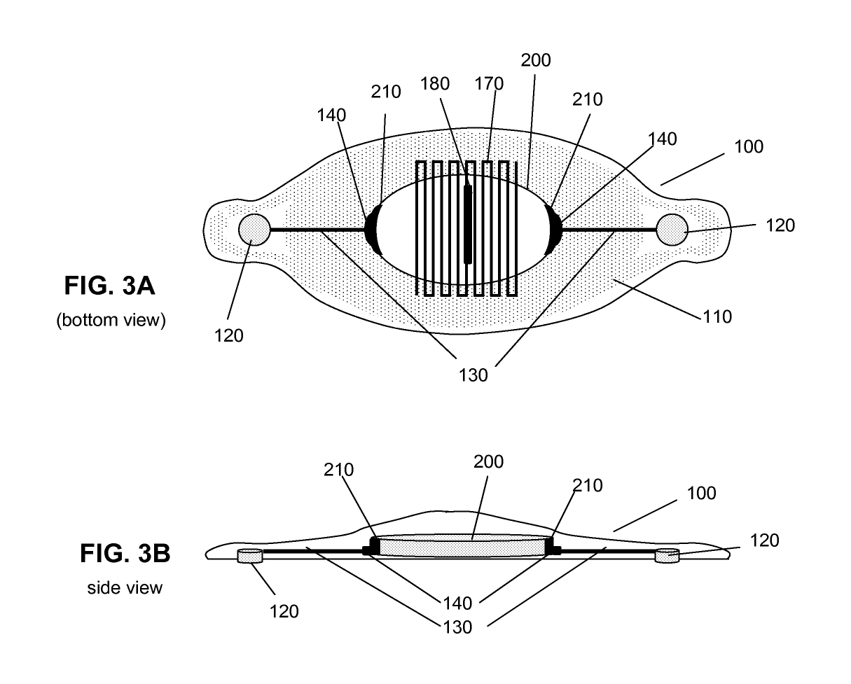

The disposable unit 100 has a hollow chamber 150, the size of which matches the size of the electronic controller 200. As illustrated in FIGS. 2A-2B, the disposable unit 100 has a cover 160 over the top (FIG. 2A) or the bottom (FIG. 2B). The cover 160 is made of a flexible biocompatible polymer and can be opened to expose the chamber 150 or closed to tightly seal the chamber 150. The electronic controller 200 can therefore be conveniently placed inside or removed from the chamber 150 of the disposable unit 100. FIGS. 3A-3B illustrates the bottom view (FIG. 3A) and the side view (FIG. 3B) of the disposable unit 100 with its chamber 150 housing the electronic controller 200. Preferably, the chamber 150 is configured to complementarily receive the electronic controller 200 so that the controller 200 is secured in its place after it is snugly fit inside the chamber 150. The electrical conducting ports 210 on the surface of the electronic controller 200 make secure contact with the respective electrical conducting terminals 140 embedded inside the disposable unit 100, so that they are electrically connected to the respective electrodes 120. In addition, the antenna interface 220 on the surface of the electronic controller 200 makes secure contact with the conducting port 180 of the wire antenna 170 embedded inside the disposable unit 100, so that the electronic controller 200 electrically connects with the wire antenna 170.

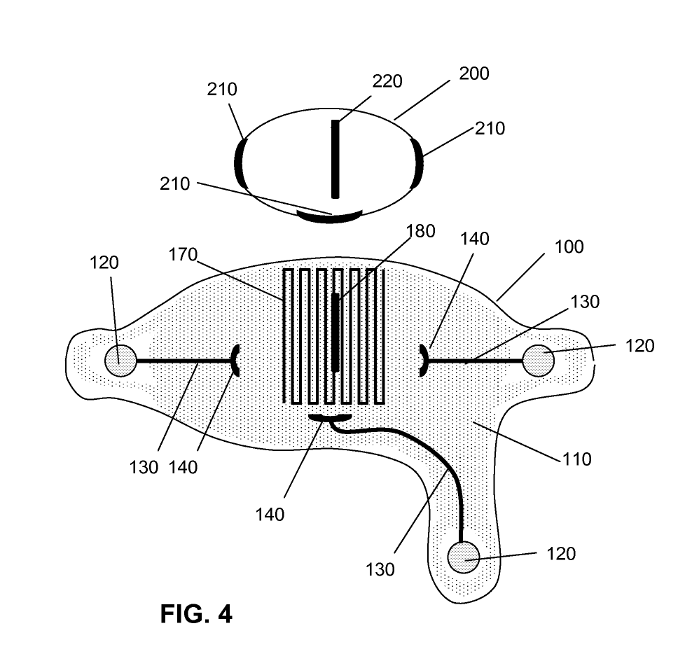

The foregoing design can be easily extended to multi-electrode configuration. For example, FIG. 4 shows the bottom view of the disposable unit 100 which has three built-in electrodes 210, a built-in wire antenna 170 that has a conducting port 180, and the corresponding electronic controller 200 which has three electrical conducting ports 210 on its surface, as well as an antenna interface 220. The geometric shape of the disposable unit 100 and the topographic distribution of the built-in electrodes 120 can be predefined or custom-designed to fit different torso shapes of the patients and to optimize the ECG recordings (for example, selection of different ECG lead vectors, increase the signal-to-noise ratio of ECG signals, etc.).

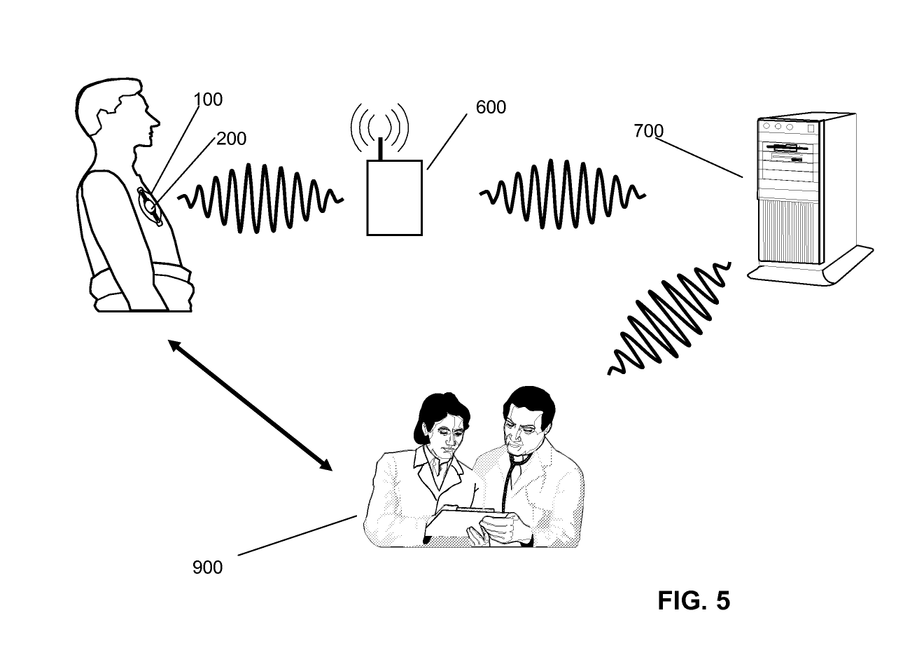

FIG. 5 schematically illustrates the preferred operation of the long-term cardiac monitoring system. The patient fits the electronic controller 200 into the disposable unit 100 which is affixed to the patient's chest, preferably over the heart. The electronic controller 200 continuously monitors the impedance between its electrical conducting ports 210 which are connected to the electrodes 120 after fitting the electronic controller 200 into the disposable unit 100. The impedance value is high (usually >1 MΩ) when the electrodes 120 are isolated from each other. When the disposable unit 100 is affixed to the patient's skin, the body tissue forms an electrical conductor. Thus, the electronic controller 200 can immediately detect the drop of the impedance value to the predefined normal range and then automatically start a self-initialization process for cutaneous ECG monitoring.

The electronic controller 200 acquires the cutaneous ECG signal measured between the electrodes 120 embedded in the disposable unit 100, analyzes the cutaneous ECG signal, and automatically triggers recording of the ECG episode upon detection of abnormal cardiac rhythm. The patient can alternatively or additionally manually trigger the recording of an ECG episode by pressing a button or switch on the external portable communication unit 600, which then sends a command to the electronic controller 200 to initiate the episode recording. Alternatively, the manual trigger for the episode recording can be placed on the disposable unit 100 or the outer surface of the electronic controller 200.

The external portable communication unit 600 can establish and maintain bi-directional wireless communications with the electronic controller 200. Through this communication link, the external portable communication unit 600 can both send commands to and retrieve diagnostic information from the electronic controller 200. The external portable communication unit 600 also can establish and maintain bi-directional communications with the remote service center 700 through a wired or wireless Home Monitoring network, through which the diagnostic information retrieved from the electronic controller 200 can be relayed to the remote service center 700 and reviewed by the patient's health care providers 900.

In a typical arrangement, the cutaneous ECG episodes and related diagnostic information recorded by the electronic controller 200 are periodically transmitted to the remote service center 700 (e.g., every midnight). In another typical arrangement, the recorded ECG episodes and related diagnostic information are immediately transmitted to the remote service center 700 upon a patient trigger, and/or upon automatic detection of predefined severe cardiac conditions (e.g. long-lasting atrial fibrillation, very high or very low ventricular rate, etc.). In another arrangement, the recorded data are transmitted to the remote service center 700 when the memory buffer of the electronic controller 200 is filled to a predefined percentage of its capacity (e.g. 80%). Preferably, after data transmission, the electronic controller 200 clears the data memory so that no data is lost due to memory saturation, and the electronic controller 200 continues ECG monitoring and data logging thereafter.

During ambulatory cardiac monitoring, the electronic controller 200 continues to monitor the impedance between the electrodes 120 as well as the signal quality of the cutaneous ECG. When the electronic controller 200 detects a fall in the impedance value to outside the predefined normal range, it generates a warning signal which is sent to the external portable communication unit 600, which then alerts the patient via perceptible means (e.g., via visual, audio, vibration, etc. signals). In addition, the electronic controller 200 also continuously evaluates the signal quality of the acquired cutaneous ECG signal. If the signal-to-noise ratio (SNR) of the ECG signal has significantly degraded, then a warning signal can also be generated and the patient is alerted.

The patient alert function is useful for long-term cutaneous cardiac monitoring. For example, loose electrode-skin contact can cause higher than normal impedance, and sweat or water may partially short circuit the electrodes and cause lower than normal impedance. After receiving the automatically generated alert of abnormal impedance or low SNR, the patient can reposition the disposable unit 100 over the chest until the impedance value between electrodes is within the normal range and the cutaneous ECG has sufficiently high SNR.

If the disposable unit 100 wears out, the patient can choose to remove the electronic controller 200 from the old disposable unit 100 and place it in a new disposable unit 100 to continue cardiac monitoring. The patient can be reminded to change the disposable unit 100 when the electronic controller 200 detects abnormal impedance between electrodes and/or low SNR of the acquired ECG and generates an alert through the external portable communication unit 600. Alternatively or additionally, the external portable communication unit 600 can be configured by the patient to set a periodic alarm (e.g. every 7 days) as a reminder for changing the disposable unit 100. Therefore, the automatic alert feature can greatly improve the patient's regular changing of the disposable unit 100, which is essential for successful long-term cutaneous cardiac monitoring.

For the purpose of long-term cardiac monitoring, it is desirable that the recording ECG lead vectors remain stable, or at least they should be well-defined, so that appropriate interpretation of the ECG can be made. This requirement may pose a special challenge when the patient changes the disposable unit 100. For example, the patient may choose to apply the new disposable unit 100 to a different chest location than the old one to prevent skin irritation from repeated use. In another example, the patient may intend to replace the disposable unit 100 at the same chest location, but the actual location and/or orientation of the new disposable unit 100 may still differ from the previous one because of human error, even if the patient is aided with a graphical guide. Therefore, the invention preferably provides quantitative feedback to the patient to guide the proper replacement of the disposable units as described hereinafter.

In a preferred version of the invention, the electronic controller 200 maintains a representative ECG beat template, i.e., a characterization of the ECG of one or more representative normal (non-irregular) heartbeats of the patient. As known in the art, the representative ECG beat template can be obtained by averaging multiple (e.g. 8, 16, etc.) normal beats of ECG signals aligned with one or more predefined fiducial points (e.g. the peak of QRS complex). Abnormal beats (such as ectopic beats or cardiac cycles during cardiac arrhythmia) are automatically detected and excluded from beat averaging. The electronic controller 200 preferably starts constructing the representative ECG beat template immediately upon affixing the disposable unit on the patient's skin, and continuously updates the representative ECG beat template based on the acquired ECG signal.

In a preferred version, after switching to a new disposable unit 100, the patient can use the portable communication unit 600 to interrogate the electronic controller 200 to check the morphology and quality of the acquired ECG signal. Typically, the latest representative ECG beat template (at the time of interrogation) is wirelessly transmitted from the electronic controller 200 to the portable communication unit 600. The newly acquired representative ECG beat template is then compared to the old representative ECG beat templates pre-stored in the portable communication unit 600.

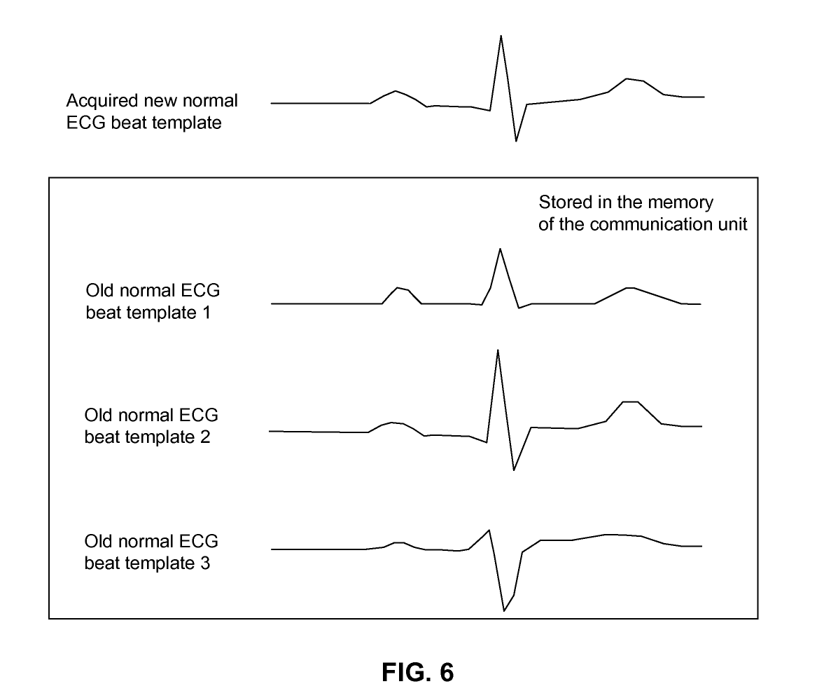

As illustrated in FIG. 6, the portable communication unit 600 typically stores multiple (preferably at least 3) old representative ECG beat templates. Typically, these old representative ECG beat templates are first generated when the patient was seen by the physician or nurse. The physician or nurse can place the disposable unit 100 in multiple locations and/or orientations to select the preferred set of ECG leads that are believed to have good signal quality, and reveal important diagnostic features from the ECG waveform. For each preferred ECG lead position, a corresponding representative ECG beat template is retrieved from the electronic controller 200 and stored in the portable communication unit 600.

As described above, after changing the disposable unit 100, the patient can use the portable communication unit 600 to interrogate the electronic controller 200 to retrieve the newly acquired representative ECG beat template. In a preferred arrangement, the morphological similarity between the newly acquired representative ECG beat template and each of the old representative ECG beat templates that are stored in the portable communication unit 600 is respectively evaluated. One typical method to measure the morphological similarity is to calculate the correlation coefficient between the new and old representative ECG beat templates. Another method to quantify the morphological similarity is to calculate the adaptive signed correlation index between the new and old representative ECG beat templates, as disclosed in U.S. Pat. Appl'n. Publ'n. 2009/0240300 A1 (which is incorporated herein by reference).

In a typical arrangement, the newly acquired representative ECG beat template is considered acceptable only when its morphology is sufficiently similar to at least one of the old representative ECG beat templates stored in the portable communication unit 600; for example, where the correlation coefficient is greater than a predefined threshold, e.g. 0.85. The location and orientation of the disposable unit 100 can be considered to be similar to that of the ECG lead corresponding to the old representative ECG beat template that results in the highest morphological similarity, and this information could then be logged into the memory of the portable communication unit 600 and further communicated to the remote service center 700. For example, as illustrated in FIG. 6, if the correlation coefficients between the newly acquired representative ECG beat template and each of the three old representative ECG beat templates are (1) 0.70, (2) 0.90, and (3) 0.30, then the disposable unit 100 is believed to have similar lead location and orientation as the ECG lead that was used to generate the old representative ECG beat template (2).

In another typical arrangement, the newly acquired representative ECG beat template is considered acceptable only when it has sufficiently high signal quality, for example, where the signal to noise ratio (SNR) of the QRS complex in the ECG beat template is greater than a predefined threshold, e.g. 20 dB. Other signal quality measures, such as the SNR of a P wave or a T wave, can also be evaluated based on the need of the ECG interpretation.

In yet another typical arrangement, the newly acquired representative ECG beat template is considered acceptable only when its morphology is sufficiently similar to at least one of the old representative ECG beat templates, and if it also has sufficiently high signal quality as measured by the SNR. On the other hand, if the newly acquired representative ECG beat template is considered not acceptable, for example because it has different morphology than the stored old representative ECG beat templates and/or if its SNR is too low, then the patient is advised to reposition the disposable unit.

In one version of the invention, the old representative ECG beat templates stored in the portable communication unit 600 are fixed after they are initially generated by the physician or nurse. In another version, the newly acquired representative ECG beat template—once it is considered acceptable (e.g., based on morphological analysis and signal quality assessment as described above)—can also be saved in the portable communication unit 600, and becomes an old representative ECG beat template, or updates it (as by being averaged with the prior representative ECG beat template), so that it can be used for comparison with later acquired new representative ECG beat templates. This dynamic update of the old representative ECG beat template is useful when the representative ECG morphology changes over time, for example due to the progression of heart diseases.

Therefore, quantitative feedback, such as the morphological similarity between the acquired ECG waveform and the pre-stored ECG templates, as well as the signal-to-noise ratio, can be provided to the patient to guide the proper placement of the disposable unit 100 on the body's surface. By this means, the ECG lead location/orientation of the disposable units 100 during the long-term monitoring period will be consistent, or at least their changes will be acceptable (in terms of signal quality and morphology), and are known to the physician or nurse who can then properly interpret the recorded ECG.

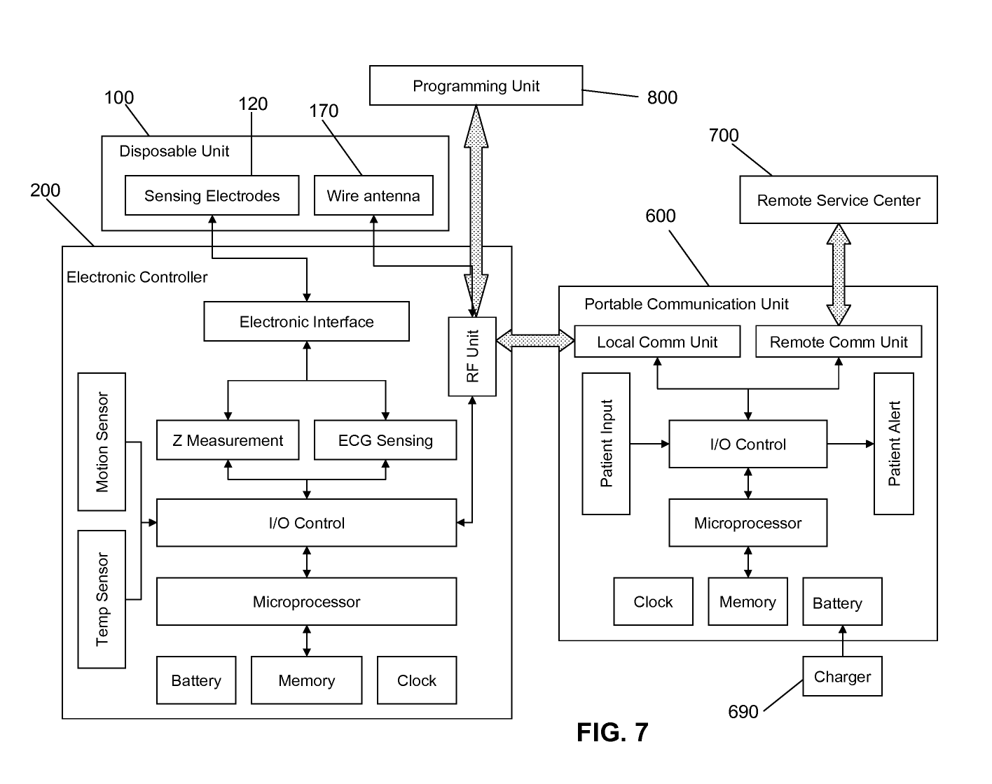

FIG. 7 shows a block diagram of an electronic controller 200 connecting with a disposable unit 100 for cutaneous ECG monitoring, and its interfaces with an external programming device 800 and a portable communication unit 600 which further communicates with the remote service center 700.

The electronic controller 200 includes electronic circuitry that is hermetically sealed inside a case. Enclosed inside the case, a microprocessor and associated circuitry make up the controller of the unit. The electronic controller 200 is powered by a battery which can preferably last at least one year in normal operation without the need for recharging, and it maintains an internal clock for timing the operations. The microprocessor communicates with a memory via a bi-directional data bus. The memory typically includes a ROM or RAM for program storage and a RAM for data storage.

By establishing the physical contact between the electrical conducting ports 210 on the case of the electronic controller 200 and the electrical conducting terminals 140 embedded in the disposable unit 100, the sensing electrodes 120 of the disposable unit 100 are electrically connected to an electronic interface of the electronic controller 200. The electronic interface preferably includes a feedthrough circuitry for noise reduction, a high voltage protection circuitry, a switch network circuitry for sensing channel selection, and front-end analog filters, as well-known in the art. The configurations of the interface circuitry (e.g. filter settings, sensing channel selection, etc.) can be programmed by the microprocessor.

In addition, by establishing the physical contact between the antenna interface 220 on the case of the electronic controller 200 and the conducting port 180 of the wire antenna 170 embedded inside the disposable unit 100, the RF unit of the electronic controller 200 electrically connects with the wire antenna 170.

The microprocessor connects to an I/O control unit to manage the input and output of the electronic controller 200. One input signal is the cutaneous ECG picked up by the sensing electrodes. After being pre-processed by the interface circuitry, the cutaneous ECG signal is further processed by the ECG sensing unit which usually includes amplifiers, analog-to-digital converters, digital filters, etc. as known in the art.

Another input signal is the impedance (Z) signal measured between the sensing electrodes 120. By injecting a small constant current (e.g., 100 μA, preferably biphasic) between two electrodes while measuring the voltage difference between the same or different pair of electrodes, the impedance is calculated as the ratio between the measured voltage difference and the injecting current strength. The impedance signal provides useful information on the integrity of the sensing channel. For example, when the sensing electrodes are in proper contact with the patient's skin, the measured Z will remain in a stable range, typically from several hundred to a few thousand ohms, depending on the materials and surface area of the sensing electrodes, the frequency of the injecting current, etc. Larger than normal Z may indicate that there is loose contact between the sensing electrodes and the patient's skin. Smaller than normal Z may indicate a short-circuit between the sensing electrodes 120, for example due to patient sweating. In addition, the continuously measured impedance signal may be further processed by the microprocessor to extract other indicia of the physiological status of the patient, such as the respiratory rate.

Other types of biological signals measured by specific sensors can also serve as inputs to the electronic controller. For example, an on-board accelerometer can serve as a motion sensor that provides patient's activity signal to the electronic controller, and an on-board temperature sensor can provide the cutaneous temperature signal to the electronic controller. Other types of input signals include, but are not limited to, the cutaneous oxygen saturation signal measured by an optical sensor, the heart sound signal measured by an acoustic sensor, etc.

By running the program stored in the memory, the microprocessor also sends instructions to the ECG sensing unit, the impedance measurement unit, and other input measurement units to control how these signals are acquired (e.g., gain, offset, filter settings, sampling frequency, sampling resolution, etc.).

The acquired biological signals are then stored in the device memory according to predefined storage modes. One typical mode is the queue-loop mode, meaning the acquired signals are stored in a predefined memory space, and while the allocated memory space is full, the newly acquired signals replace the oldest stored data. Another typical mode is the snapshot mode, meaning the acquired signals are stored in a predefined memory space, and while the allocated memory space is full, the newly acquired signals are not stored until the microprocessor decides to store another episode of data. Yet another typical mode is the mixed mode, in which one or more segments of allocated memory space store the acquired signals in queue-loop mode, whereas one or more segments of separately allocated memory space store the data in snapshot mode.

The acquired biological signals are analyzed by the microprocessor by running programmed algorithms. For example, the microprocessor continuously analyzes the acquired cutaneous ECG signals to detect the peak of the QRS complex. Accordingly, the electronic controller measures the intervals between any two adjacent peaks of the detected QRS complexes, and these intervals are termed RR intervals. These measured RR intervals are stored in the device memory. Similarly, the microprocessor can also continuously analyze the acquired cutaneous ECG signals to measure other metrics of the QRS complex, such as the width of the QRS complex, the positive or negative peak amplitude of the QRS complex, the absolute area under the QRS complex, the maximum positive or negative slopes of the QRS complex, the dominant frequency component of the QRS complex, the complexity measures of the QRS complex, and so on. Likewise, the time series of these measured metrics are stored in the device memory for further analysis.

The electronic controller also includes a radio-frequency (RF) telemetry unit. The RF telemetry unit may be of the type well-known in the art for conveying various information, which it obtains from the electronic controller, to the external programmer, or for receiving programming parameters from the external programmer and then conveying it to the electronic controller. In one typical arrangement, the external programmer can interrogate the electronic controller and get its working status (e.g., battery status, sensing channel impedance, etc.) or the data recorded by the electronic controller (e.g. peak amplitude of the QRS complexes, statistics of measured RR intervals, etc.). In another typical arrangement, the external programmer can be used to activate or deactivate selected algorithms or update programmable parameters of the electronic controller.

In addition, the external portable communication unit to be described hereinafter can also communicate bi-directionally with the electronic controller through the telemetry unit. Preferably, the data that may be received from or sent to the external portable communication unit are more limited compared to the data that may be received from or sent to the external programmer.

In a preferred version, the data that are transmitted from the external portable communication unit to the electronic controller are simple commands, such as a command to trigger a snapshot of the acquired cutaneous ECG, to retrieve the most recently diagnostic information from the electronic controller, etc. These commands set the electronic controller into one of a number of modalities, wherein each modality is determined and controlled by parameters that can only be selected by a physician operating the external programmer using secure password or codes.

The data that are transmitted from the electronic controller to the external portable communication unit preferably include a simple acknowledgment to confirm receiving the commands from the external portable communication unit, and signals warning of the detection of abnormal conditions, such as detection of atrial fibrillation (AF), detection of high or low ventricular rate, detection of abnormal sensing impedance, detection of abnormal temperature, and so on. Other diagnostic information, such as the AF burden, the frequency of ectopic beats, snapshots of RR intervals or cutaneous ECG, etc. can also be transmitted to the external portable communication unit. Preferably, a physician operating the external programmer (using secure password or codes) controls the enable or disable condition, as well as the amount of data that can be transmitted from the electronic controller to the external portable communication unit.

Again referring to FIG. 7, the external portable communication unit has a power source, such as a lithium battery, which provides power to the electrical components of the unit. The battery is chargeable by connecting to an external charger. The external portable communication unit also maintains an internal clock for timing its operations. The overall functioning of the external portable communication unit is controlled by its microprocessor, which reads and performs instructions stored in its associated memory. The instructions stored in memory preferably include instructions defining a communication protocol compatible with the electronic controller, and instructions defining a communication protocol compatible with the remote service center.

The microprocessor of the external portal communication unit communicates with an I/O control unit to read patient input commands from the keypad (or other input device). In an exemplary version, one subset of the input commands is designed to configure the external portable communication unit, for example to turn on or off certain outputs as described hereinafter, or select specific communication protocols. Another subset of the input commands might establish communication between the external portable communication unit and the remote service center through the remote communication unit. For example, the patient's input can command the external portable communication unit to transmit diagnostic information (retrieved from the electronic controller) to the remote service center and wait to receive an acknowledgment. Another subset of the commands might establish communication between the external portable communication unit and the electronic controller through the local communication unit. For example, the patient's input can command the external portable communication unit to transmit corresponding signals to the electronic controller to trigger recording a snapshot of the cutaneous ECG, to retrieve diagnostic information from the electronic controller, etc. The local communication unit also receives the acknowledgment and related diagnostic information sent from the electronic controller, and conveys these data to the microprocessor for storage in the memory.

In an exemplary version of the invention, upon receiving a predefined warning signal from the electronic controller (e.g., detection of AF, detection of high or low ventricular rate, detection of abnormal sensing impedance, detection of abnormal temperature, etc.), the microprocessor of the external portable communication unit communicates with the I/O control unit to generate output that is perceptible by the patient. Such output can be in the form of (for example) a visible message such as the illumination of a light emitting diode (LED) or a text message displayed in a liquid crystal display (LCD), or in the form of audible message such as a beep, ringing tone or pre-recorded voice message played by a speaker, or in the form of discernible vibration by a vibrator. According to the patient's preference, one or multiple types of warning messages can be respectively turned on or off. For example, the visible warning message can be turned on while the audible warning message can be turned off during the night if the patient chooses not to be disturbed during sleep, even if the electronic controller detects AF. Besides generating warning messages, some diagnostic information that is received from the electronic controller and stored in memory (e.g., the heart rate) can also be provided to the patient in the form of visual or audible messages.

The external portable communication unit, via its remote communication unit, can further communicate with the remote service center. Such a long-range communication device can be in the form of a mobile radio network, a fixed-line telecommunication network, or the internet, as well known in the art. Examples of such long-range communication devices have been taught in U.S. Pat. No. 6,470,215, U.S. Pat. No. 6,574,509, U.S. Pat. No. 6,622,043, all of which being incorporated herein by reference.

In a typical version, the external portable communication unit transmits the electronic controller status information (e.g. battery status, sensing impedance, etc.) as well as relevant diagnostic information (e.g. AF burden, ectopic beat frequency, etc.) to the remote service center according to a predefined transmission frequency and schedule (e.g. every midnight, etc.). In another typical arrangement, the external portable communication unit communicates with the remote service center in a trigger mode, for example upon receiving a warning signal from the electronic controller, or upon receiving the patient trigger, or when the filled memory buffer of the electronic controller reaches a predefined percentage of its capacity. In such cases, the external portable communication unit transmits critical diagnostic information stored in the device memory (e.g. AF burden, mean heart rate, the ECG snapshot, etc.) to the remote service center.

The remote service center receives the information via compatible communication protocols, then sends an acknowledgment back to the portable communication unit, which may generate visible or audible output indicating receipt of the acknowledgment. The data received by the remote service center is stored in a secured central database and is promptly presented to the patient's physician or another responsible expert through proper means, such as email, text, and/or fax messaging, as known in the art. By reviewing the received diagnostic information, the physician can evaluate the patient's condition and provide expert advice to the patient, who may wish to contact the physician before taking any action in response to the warning signals generated by the external portable communication unit.

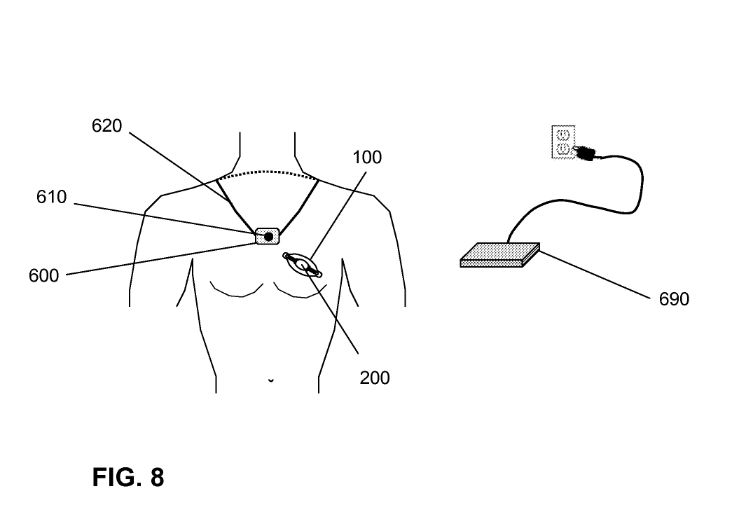

The external portable communication unit is designed to be easily carried by the patient. For example, it can be carried in a pocket, clipped on a belt, or worn like a watch or a necklace, etc. As an example, FIG. 8 shows the external portable communication unit 600 having a loop antenna 620 designed in the form of a necklace. FIG. 8 also shows that the patient is wearing a disposable unit 100 which encloses an electronic controller 200 for cutaneous cardiac monitoring. The long antenna length encircles a loop area which forms a large effective aperture. In addition, the external portable communication unit 600 is close to the electronic controller 200, and their spatial distance and orientation are relatively stable. All these factors favor more reliable communications between the external portable communication unit 600 and the electronic controller 200.

FIG. 8 also shows that the external portable communication unit 600 has a button 610 which can be pressed by the patient to trigger an event recording by the electronic controller 200. The recorded event, along with related diagnostic information stored by the electronic controller 200, is first transmitted to the external portable communication unit 600, which then relays the information to the remote service center through the wired or wireless Home Monitoring network.

The portable communication unit 600 can be recharged on a charging station 690. As known in the art, the charging can be implemented in either conductive charging (i.e. direct electrical contact between the battery and the charger) or inductive charging (i.e. use of an electromagnetic field to transfer energy between the battery and the charger). During charging, the portable communication unit 600 can still communicate with the electronic controller 200, as long as their distance is within a specified range, e.g. 6 feet. Preferably, the portable communication unit 600 can be powered by a backup battery while its is rechargeable battery is recharged in the charging station 690. Hence, patient can still wear the fully powered portable communication unit 600 while its rechargeable battery is recharged.

Multiple mechanisms can be designed to improve the patient appliance for recharging the battery. For example, when the portable communication unit 600 detects its battery voltage is below a predefined value, a warning signal in various forms, such as audible beeps, vibrations, etc. may be generated to alert the patient. In another example, the charging station 690 has a user-programmable unit that allows patient to set a timed alarm (e.g. every night at 9 p.m.) as a reminder for charging the battery.

The disclosed system, and its component devices and methods of operation, provide a useful solution for long-term non-invasive cardiac monitoring. A patient can replace the disposable units as often as needed while using the same electronic controller and the portable communication units, which preferably operate for at least a year. No battery recharging is necessary for the electronic controller, thus causing little disruption to the cardiac monitoring. Besides conventional ECG monitoring capabilities, the automatic alert features (e.g. detection of skin-electrode contact and signal quality) can greatly improve patient compliance, which is essential for long-term monitoring. In conjunction with the Home Monitoring feature, this system reduces the risk of memory saturation, which is a common problem for most existing cardiac monitors. Moreover, quantitative feedback is provided to the patient on the quality of the acquired ECG signal to guide the proper replacement of the disposable units.

Further advantages such as the potentially low cost of the system, the convenience of its usage, combined with the high diagnostic yield, represent high added value.

Although exemplary versions of the invention have been shown and described, it should be apparent to those of ordinary skill that numerous modifications and variations are possible. The disclosed examples are presented for purposes of illustration only, and other versions of the invention may include some or all of the features disclosed herein. Therefore, the invention is not limited to the versions of the invention described above, but rather is limited only by the claims set out below, with these claims securing rights to all different versions that fall literally or equivalently within the scope of these claims.

What is claimed is:

1. A system for long-term cutaneous cardiac monitoring including:
A. a disposable unit configured to be adhered to a patient's skin, and
B. an electronic controller detachably connected to the disposable unit,
wherein:
a. the disposable unit includes:
(1) a substantially flat skin-contacting portion having pick-up electrode poles situated to electrically contact a patient's skin at spaced-apart locations,
(2) an antenna; and
(3) an internal chamber:
(a) attached to the skin-contacting portion,
(b) configured to accommodate the electronic controller therein in a watertight sealed manner,
(c) configured to open and close so as to allow insertion of the electronic controller therein, and removal therefrom,
(d) including electrode terminals therein that are electrically connected to the pick-up electrode poles, and
(e) including an antenna port therein that is electrically connected to the antenna; and
b. the controller:
(1) includes electrical conducting ports situated to make conductive contact with the electrode terminals and the antenna port when the controller is inserted within the chamber, thereby establishing electrical contact with the pick-up electrode poles and the antenna,
(2) is configured to acquire, process and store physiological signals acquired via the pick-up electrode poles.

2. The system of claim 1 wherein the controller further includes a battery having a capacity sufficient to allow the controller to operate for at least one year.

3. The system of claim 1 wherein the controller is configured to provide a quantitative measure of the signal quality of a signal acquired via the pick-up electrode poles.

4. The system of claim 1 wherein the controller is further configured to store a representative ECG beat template.

5. The system of claim 4 wherein the controller is further configured to construct the representative ECG beat template by averaging multiple normal beats of ECG signals:
a. acquired via the pick-up electrode poles, and
b. being aligned with a predefined fiducial point.

6. The system of claim 4 wherein the controller is further configured to:
a. start constructing the representative ECG beat template immediately upon affixing the disposable unit on a patient's skin, and
b. continuously update the representative ECG beat template based on acquired ECG signals.

7. The system of claim 1 wherein the electrode poles are spaced apart by an inter-electrode distance of at least 3 cm.

8. The system of claim 1 wherein the controller includes:
a. an ECG sensing unit, and
b. a memory,
hermetically sealed within an outer controller case.

9. The system of claim 1 wherein the controller includes an impedance measuring unit:
a. connected to the conducting ports, and
b. configured to measure an impedance signal between the electrode poles.

10. The system of claim 1 further including a portable communication unit configured to wirelessly communicate bi-directionally with:
a. the controller via the antenna, and
b. a remote service center.

11. A system for long-term cutaneous cardiac monitoring including:
a. a disposable unit including:
(1) an openable and closable chamber, the chamber being waterproof when closed,
(2) an adhesive surface,
(3) electrodes situated to be in contact with the skin of a patient to which the adhesive surface is adhered, and
(4) an antenna,
b. an electronic controller configured to:
(1) fit into the chamber,
(2) establish electrical contact with the electrodes and antenna when fit into the chamber, and
(3) acquire, process and store signals from the electrodes, and
(4) wirelessly transmit signals from the antenna.

12. The system of claim 11 further including a battery powering the controller, wherein the battery has a battery life of at least 1 year.

13. The system of claim 11 further including a portable communication unit configured to wirelessly communicate bi-directionally with:
a. the controller via the antenna, and
b. a remote service center.

14. The system of claim 13 wherein the portable communication unit is configured to obtain from the controller information regarding:
a. the morphology, and
b. the quality,
of an ECG signal acquired from the electrodes.

15. The system of claim 13 wherein the controller is configured to:
a. wirelessly transmit a latest representative ECG beat template to the portable communication unit, and
b. thereafter compare a newly acquired representative ECG beat template to one or more old representative ECG beat templates stored in the portable communication unit.

16. The system of claim 13 wherein:
a. at least one of the portable communication unit and the controller is configured to rate the acceptability of a newly acquired representative ECG beat template, and
b. the acceptability rating is determined in accordance with the degree of correspondence between:
(1) the morphology of the newly acquired representative ECG beat template, and
(2) the morphology of at least one of the old representative ECG beat templates stored in the portable communication unit.

17. The system of claim 16 wherein at least one of the portable communication unit and the controller is configured to generate a signal indicating non-optimal placement of the disposable unit when the newly acquired representative ECG beat template is rated unacceptable.

18. The system of claim 13 wherein:
a. at least one of the portable communication unit and the controller is configured to rate the acceptability of a newly acquired representative ECG beat template, and
b. the acceptability rating is determined in accordance with a signal quality measurement determined for the newly acquired representative ECG beat template.

19. The system of claim 18 wherein the acceptability rating is also determined in accordance with the degree of correspondence between:
a. the morphology of the newly acquired representative ECG beat template, and
b. the morphology of at least one of the old representative ECG beat templates stored in the portable communication unit.

20. A system for long-term cutaneous cardiac monitoring, the system including:
a. a disposable patient-mounted electrode unit including:
(1) a skin-contacting surface bearing spaced pick-up electrode poles configured to electrically contact a patient's skin when the skin-contacting surface is situated on the patient's skin,
(2) an antenna; and
(3) an internal chamber including:
(a) electrode terminals therein, the electrode terminals being in conductive communication with the pick-up electrode poles, and
(b) an antenna port therein, the antenna port being in conductive communication with the antenna; and
b. a controller configured:
(1) to be removably fit into the internal chamber,
(2) to establish conductive contact with the electrode terminals, and acquire and process signals from the pick-up electrode poles, when the controller is removably fit into the internal chamber,
(3) to establish conductive contact with the antenna port, and wirelessly transmit signals from the antenna, when the controller is removably fit into the internal chamber.
